(19) United States
(12) Patent Application Publication
(10) Pub. No.: US 2023/0001151 A1
(43) Pub. Date: Jan. 5, 2023

(54) THORACIC CATHETER PROTECTOR POUCH

(71) Applicants: Karen Kagan, Fort Myers, FL (US); Gabriel Kagan, Fort Myers, FL (US); Evan Kagan, Fort Myers, FL (US)

(72) Inventors: Karen Kagan, Fort Myers, FL (US); Gabriel Kagan, Fort Myers, FL (US); Evan Kagan, Fort Myers, FL (US)

(21) Appl. No.: 17/366,399

(22) Filed: Jul. 2, 2021

(51) Int. Cl. A61M 25/02

(52) U.S. Cl. CPC A61M 25/02; A61M 2210/10; A61M 2205/04; A61M 2205/0216; A61M 2025/026

(57) ABSTRACT

A thoracic catheter protector pouch (1) to be worn by an adult, person or infant has a band (2) that encircles a body portion, such as a chest or arm, and a medial pocket portion (5) for holding a catheter tube (10) and connector end (11). The pocket portion has an inner panel (15) with a catheter tube securement strap (14) and two flaps (3 and 4) which open to insert a catheter and close over each other securely by fasteners (6 and 7) preferably hook and loop fastening material. The band is preferably elastomeric and is size adjustable.

### BACKGROUND OF THE INVENTION

The present invention relates to a thoracic catheter protector pouch, and, more particularly, to a catheter protector pouch to prevent injury to a catheter entry site into a patient's body and avoid a catheter from being pulled out from the entry site.

The conventional method still being used to this day to secure the external portion of implanted catheters to the skin of a patient is to employ medical tape. Unfortunately, this conventional method is fraught with problems. One problem is that the tape can be inadvertently or intentionally removed by the patient prematurely thereby leaving the catheter unsecured and exposed from the insertion site in the skin where it enters the body. The unsecured catheter is also susceptible to accidental premature removal which requires replacement of a new catheter which requires a surgical procedure performed by a hospital. Additionally, another problem results from the necessarily repeated cleaning and disinfection of the catheter entry site into the body, thereby requiring frequent removal and reapplication of medical tape on the sensitive skin of patients, especially infants and young persons, which causes itching, severe irritation and soreness.

Attempts to eliminate the need for taping have led to the development of covers for enclosing and protecting the external portion of the catheter and which are affixed to the patient's skin with a less irritating medical grade adhesive similar to but less irritating than that employed by regular medical tape. Devices of this sort are undesirable to the catheter patient because the use of any adhesive over time results in the same type of skin irritation and soreness associated with the use of regular medical tape. Furthermore, the adhesive used with these devices typically employs lower adhesion capability causing more frequent premature departure from the skin.

None of the previous devices have adequately protected both the external segment of the catheter and the entry site into the body from these problems giving rise to increased risks of serious injury among adults and especially infants and young persons having implanted catheters.

Thus, a need exists for an apparatus which not only protects the external end of a catheter and secures it to the body, but which provides a means for protecting the entry site from the sources of injury described above.

Other past attempts to resolve this issue include the following references, but none disclose a thoracic catheter protective pouch like the present invention:

| Patent/App. No. | Inventor | Issue/Pub. Date |
|---|---|---|
| 5,755,698 | Kagan et al. | May 26, 1998 |
| 5,048,122 | Prieur | Sep. 17, 1991 |
| 6,126,639 | Sutherland et al. | Oct. 3, 2000 |
| 6,206,854 | Weaver | Mar. 27, 2001 |
| 6,579,268 | Loining | Jun. 17, 2003 |
| 6,681,404 | Adlard et al. | Jan. 27, 2004 |
| 2005/0033241 | Hottinger | Feb. 10, 2005 |
| 7,201,739 | Walborn | Apr. 10, 2007 |
| 2009/0054844 | Alyea et al. | Feb. 26, 2009 |
| 10/149,964 | Fitzgerald et al. N/A | Dec. 11, 2018 |
| D810,951 | Tooke | Feb. 20, 2018 |

### SUMMARY OF THE INVENTION

The primary object of the present invention is to provide an apparatus to retain a thoracic catheter securely in place to a patient who could be an adult or an infant.

Another object of the present invention is to provide such an apparatus that eliminates the need for continual replacement of catheters when such become dislodged during use.

The present invention fulfills the above and other objects by providing a thoracic catheter protector apparatus for affixing an exposed segment of a catheter by having an elongated fabric band for encircling the body, and covering a catheter implant site with a pocket portion on a medial portion of the pouch. When the apparatus is worn, the pocket portion has on one side a planar surface facing the body when the apparatus is worn and an exterior portion having two flaps, an inner flap and an outer flap. The inner and outer flaps form a compartment for receipt of the exposed segment of catheter. The compartment has at least one securement fastener disposed about at least one peripheral edge of the outer flap, preferably strips of hook and loop fastening material. The inner panel of the pouch may also have a securement strip or loop which may be hook and loop to hold a catheter tube more securely within the pouch. The band attached to and used to wrap the pouch around a person's body is preferably elastomeric and is size adjustable by a using a bracket and with a band overlap.

The above and other objects, features and advantages of the present invention should become even more readily apparent to those skilled in the art upon a reading of the following detailed description in conjunction with the drawings wherein there is shown and described illustrative embodiments of the invention.

### DESCRIPTION OF THE DRAWINGS

In the following detailed description, reference will be made to the attached drawings in which:

FIG. 1 is a front perspective view of the thoracic catheter protector pouch of the present invention with the catheter protective pocket in an open mode;

FIG. 2 is a front planar view of the thoracic catheter protector pouch of the present invention being worn by a person;

FIG. 3 is a rear view of the thoracic catheter protector pouch of the present invention being worn by a person;

FIG. 4 is an enlarged cross-sectional view of the thoracic catheter protector pouch of the present invention along section line 4-4 of FIG. 2 with the catheter secured within the holding pocket; and

FIG. 5 is an enlarged cross-sectional view of the thoracic catheter protector pouch of the present invention along section line 5-5 of FIG. 2 with the catheter secured within the holding pocket.

### DESCRIPTION OF THE PREFERRED EMBODIMENTS

For purposes of describing the preferred embodiment, the terminology used in reference to the numbered items in the drawings is as follows:

1. thoracic catheter protective pouch, generally

2. torso band

3. inner flap

4. outer flap

5. pocket

6. hook and loop fastening strips (inner flap)

7. hook and loop fastening strip (outer flap)

8. size adjuster

9. person

10. catheter tube

11. catheter end connector

12. inner exterior of pocket

13. band overlap

14. catheter tube securement loop

15. inner panel of pouch

16. hooked attachment bracket

17. attachment strap loop

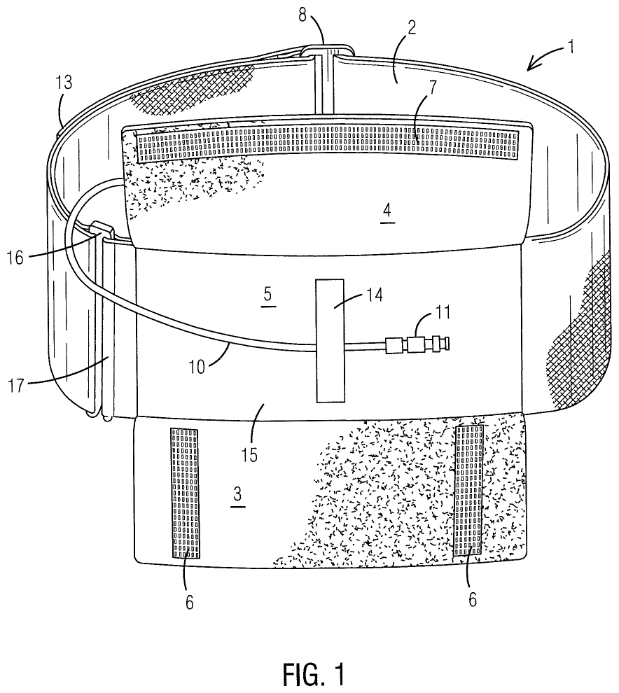

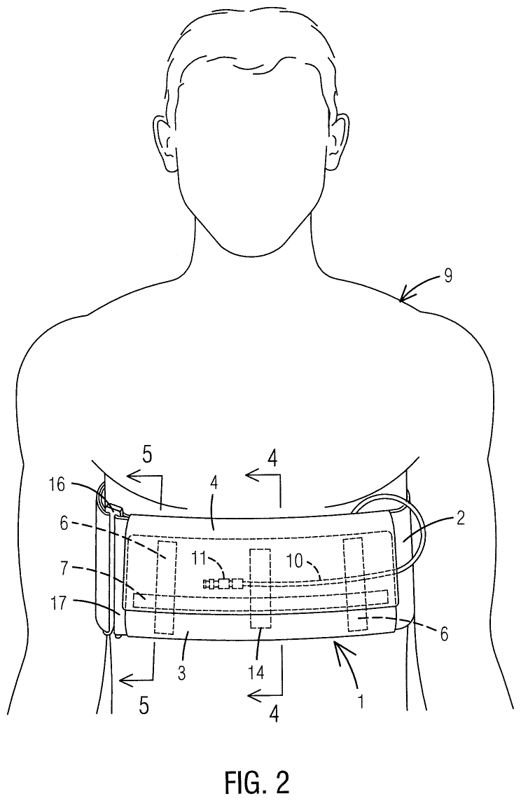

Referring now to the FIG. 1, a perspective view of the thoracic catheter protective pouch 1 of the present invention is illustrated. The thoracic catheter protective pouch 1 has a torso band 2, which is preferably made of elastomeric material and can be worn around a chest of a person 9 (as shown in FIG. 2) or another part of a torso. The band 2 is size adjustable by means of a size adjuster 8 and band overlap 13 to be lengthened or shortened in length depending on the circumference of the torso around which the thoracic catheter protective pouch 1 is placed. The catheter holding pocket 5 is integrated medially in a front of the pouch 2 and has a lower inner flap 3 and a top outer flap 4. The pocket 5 and flaps 3 and 4 are preferably made of a soft material e.g. felt for comfort and also to enable hook and loop fastening material, such as the hook and loop fastening strips 6 on inside of the inner flap 3 to attach to the pocket 5 when closed to securely hold a catheter in place. Similarly, the hook and loop fastening strip 7 on inside the outer flap 4 enable it to securely fasten on outside of the inner pocket 3 when the pocket 5 is closed. In addition, a catheter tube securement loop 14 is attached to an inner panel of the pocket 5 through which the catheter tube 10 is inserted to better secure the catheter tube in place. The catheter holding pocket 5 is removably attachable to the band 2 by an attachment strap loop 17 sewn into one end of the pocket 5 that connects to a hook attachment bracket 16 on the band 3 so as to allow the entire thoracic catheter protective pouch 1 to be easily removed from around a wearer for bathing and other reasons.

FIG. 2 is a planar view of the thoracic catheter protective pouch 1 being worn around the chest of a person 9. The pocket 5 is shown attached to medial ends of the torso band 2 in a closed mode with the inner flap 3 and outer flap 4 fastened by hook and loop fastening strips 6 and 7 over a catheter tube 10 and catheter connector end 11. The catheter tube securement loop 14 holds the catheter tube 10 in place inside the pocket 5 of the thoracic catheter protective pouch 1.

FIG. 3 is a rear perspective view of a person 9 wearing the thoracic catheter protective pouch 1 is illustrated showing the torso pband 2 and the size adjuster 8.

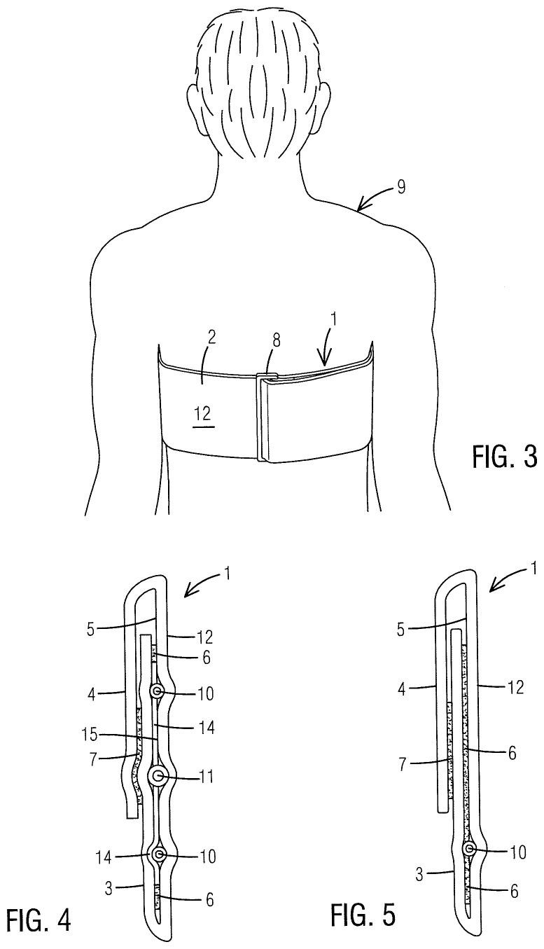

FIG. 4 illustrates the thoracic catheter protective pouch 1 in cross-sectional view along section line 4-4 of FIG. 2. In this view the catheter tube 10 and catheter connector end 11 are shown being secured within the pocket 5 by the inner flap 3 and the outer flap 4 being secured over the inner flap 3 by hook and loop fastener strip 7. The pocket 5 would preferably have an exterior side 12 made of soft material, such as felt, for comfort when worn against the skin of a person and to prevent chafing. The catheter tube 10 is held securely in place by a securement loop 14 to the inner panel 15 of the pouch 1.

FIG. 5 is a cross-sectional view of the pocket 5 along section line 5-5 of FIG. 2 showing the catheter end connector 11 being secured within the pocket 5 by the inner flap 3 being attached by hook and lop fastener strip 6 to an inner side of the pocket 5 with the outer flap 4 folded over and attached over it by hook and loop fastener strip 7.

Although several aspects have been disclosed in the foregoing specification, it is understood by those skilled in the art that many modifications and other aspects will come to mind to which this disclosure pertains, having the benefit of the teaching presented in the foregoing description and associated drawings. It is thus understood that the disclosure is not limited to the specific aspects disclosed hereinabove, and that many modifications and other aspects are intended to be included within the scope of any claims that can recite the disclosed subject matter.

Having thus described my invention, I claim:

1. A thoracic catheter protector pouch for affixing an exposed segment of a catheter implanted in a body of a patient, said pouch comprising:
an elongated fabric band for encircling a part of the body and covering a catheter implant site configured with a pocket portion on a medial portion of the band;
said pocket portion having on one side a planar surface facing the torso when the pouch is worn, an inner panel and an exterior portion having an inner flap and an outer flap;
a catheter tube securement strap on the inner panel of the pocket portion;
said inner and outer flaps forming a compartment for receipt of said exposed segment of catheter; and
said compartment being further formed with at least one securement fastener disposed about at least one of the two flaps.

2. The thoracic catheter protector pouch of claim 1 wherein:
the elongated band is made of elastomeric material.

3. The thoracic catheter protector pouch of claim 1 wherein:
the elongated band is size adjustable.

4. The thoracic catheter protector pouch of claim 2 wherein:
the elongated band is size adjustable.

5. The thoracic catheter protector pouch of claim 1 wherein:
the at least one securement fasteners on the flaps comprises hook and loop fastening material.

6. The thoracic catheter protector pouch of claim 1 wherein:
the catheter tube securement strap on the inner panel of the pocket portion comprises hook and loop fastening material.

7. The thoracic catheter protector pouch of claim 1 wherein:
the outer flap is attached along a top edge of the pocket portion and the inner flap is attached along a bottom edge of the pocket portion.

8. A thoracic catheter protector pouch for affixing an exposed segment of a catheter implanted in a body of a patient, said pouch comprising:
an elongated fabric band for encircling a part of the body and covering a catheter implant site configured with a pocket portion on a medial portion of the band;
said pocket portion having on one side a planar surface facing the torso when the apparatus is worn, an inner panel and an exterior portion having two flaps, an inner flap and an outer flap;
a catheter tube securement strap on the inner panel of the pocket portion comprised of hook and loop fastening material;
said inner and outer flaps forming a compartment for receipt of said exposed segment of catheter; and
said compartment being further formed with at least one securement fastener disposed about at least one of the two flaps; wherein
the elongated band is made of elastomeric material and is size adjustable, the at least one securement fasteners on the flaps comprises hook and loop fastening material; and
the outer flap is attached along a top edge of the pocket portion and the inner flap is attached along a bottom edge of the pocket portion.
